(19) United States
(12) Patent Application Publication
(10) Pub. No.: US 2026/0029361 A1
(43) Pub. Date: Jan. 29, 2026

(54) GAS DETECTION METHOD AND GAS DETECTION SYSTEM

(71) Applicant: Panasonic Intellectual Property Management Co., Ltd., Osaka (JP)

(72) Inventors: Takuya HAYASHI, Osaka (JP); Masaya NAKATANI, Hyogo (JP)

(21) Appl. No.: 18/994,460

(22) PCT Filed: Jul. 3, 2023
(86) PCT No.: PCT/JP2023/024561

§ 371 Date: Jan. 14, 2025

(30) Foreign Application Priority Data
Jul. 29, 2022 (JP) 2022-121973

(51) Int. Cl. G01N 27/12; G01N 33/00

(52) U.S. Cl. CPC G01N 27/124; G01N 33/0036

(57) ABSTRACT

A gas detection method includes: (a) alternating, in a first cycle, between heating and non-heating of a gas sensor exposed to a sample gas that includes a detection-target gas and a non-detection-target gas other than the detection-target gas; (b) obtaining the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and (c) multiplying, with use of a multiplier, the gas adsorption-desorption signal obtained in (b) and a reference signal that repeats a rise and a fall in a regular cycle.

### TECHNICAL FIELD

The present disclosure relates to a gas detection method and a gas detection system.

### BACKGROUND ART

Patent Literature (PTL) 1 discloses a gas sensor that detects the concentration of a specific gas component (such as oxygen or NOx) in exhaust gas emitted from a vehicle engine.

### CITATION LIST

### Patent Literature

### PTL 1

Japanese Unexamined Patent Application Publication No. 2022-59970

### SUMMARY OF INVENTION

### Technical Problem

The gas sensor disclosed in PTL 1 above, however, is hard to apply if one wants to identify a foul-smelling gas that has occurred at a foul odor source contained, for example, in a continuous flow of a detection-target gas.

Accordingly, the present disclosure provides a gas detection method and a gas detection system that each can help improve the accuracy of detecting a detection-target gas.

### Solution to Problem

A gas detection method according to one aspect of the present disclosure is a gas detection method of detecting a detection-target gas that occurs at a target item, with use of a gas sensor that outputs a gas adsorption-desorption signal corresponding to a gas adsorption concentration, and the gas detection method includes: (a) alternating, in a first cycle, heating and non-heating of the gas sensor exposed to a sample gas that includes the detection-target gas and a non-detection-target gas other than the detection-target gas; (b) obtaining the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and (c) multiplying, with use of a multiplier, the gas adsorption-desorption signal obtained in (b) and a reference signal that repeats a rise and a fall in a regular cycle.

A gas detection system according to another aspect of the present disclosure is a gas detection system that detects a detection-target gas that occurs at a target item, and the gas detection system includes: a gas sensor that outputs a gas adsorption-desorption signal corresponding to a gas adsorption concentration; a temperature adjusting device that alternates, in a first cycle, between heating and non-heating of the gas sensor exposed to a sample gas that includes the detection-target gas and a non-detection-target gas other than the detection-target gas; an obtainer that obtains the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and a multiplier that multiplies the gas adsorption-desorption signal obtained by the obtainer and a reference signal that repeats a rise and a fall in a regular cycle. It is to be noted that general or specific aspects of the above may be implemented in the form of a system, a method, an integrated circuit, a computer program, or a computer readable recording medium, such as a compact disc-read only memory (CD-ROM), or may be implemented through any desired combinations of a system, a method, an integrated circuit, a computer program, and a recording medium.

### Advantageous Effects of Invention

The gas detection method and so forth according to some aspects of the present disclosure can improve the accuracy of detecting a detection-target gas.

### BRIEF DESCRIPTION OF DRAWINGS

FIG. 1 is a diagram showing an overview of a gas detection system according to Embodiment 1.

FIG. 2 is a block diagram showing a configuration of a gas detection system according to Embodiment 1.

FIG. 3 is a diagram showing one example of waveforms of gas adsorption-desorption signals according to Embodiment 1.

FIG. 4 is a diagram showing one example of each of the waveforms of a gas adsorption-desorption signal, a gas sensor temperature, a reference signal, a multiplier output signal, and a low pass filter output signal in a gas detection system according to Embodiment 1.

FIG. 5 is a flowchart showing a flow of an operation of a gas detection system according to Embodiment 1.

FIG. 6 is a diagram showing an overview of a gas detection system according to Embodiment 2.

FIG. 7 is a block diagram showing a configuration of a gas detection system according to Embodiment 2.

FIG. 8 is a diagram showing one example of waveforms of gas adsorption-desorption signals according to Embodiment 2.

FIG. 9 is a diagram showing one example of each of the waveforms of a detection-target gas adsorption-desorption signal, a gas sensor temperature, a reference signal, a multiplier output signal, and a low pass filter output signal in a gas detection system according to Embodiment 2.

FIG. 10 is a diagram showing one example of each of the waveforms of a non-detection-target gas adsorption-desorption signal, a gas sensor temperature, a non-detection-target gas adsorption-desorption signal observed after the gas passes through a high pass filter, a reference signal, a multiplier output signal, and a low pass filter output signal in a gas detection system according to Embodiment 2.

FIG. 11 is a flowchart showing a flow of an operation of a gas detection system according to Embodiment 2.

### DESCRIPTION OF EMBODIMENTS

A gas detection method according to a first aspect of the present disclosure is a gas detection method of detecting a detection-target gas that occurs at a target item, with use of a gas sensor that outputs a gas adsorption-desorption signal corresponding to a gas adsorption concentration, and the gas detection method includes: (a) alternating, in a first cycle, between heating and non-heating of the gas sensor exposed to a sample gas that includes the detection-target gas and a non-detection-target gas other than the detection-target gas; (b) obtaining the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and (c) multiplying, with use of a multiplier, the gas adsorption-desorption signal obtained in (b) and a reference signal that repeats a rise and a fall in a regular cycle.

According to this aspect, as the gas adsorption-desorption signal and the reference signal are multiplied by the multiplier, the accuracy of detecting the detection-target gas adsorption-desorption signal included in the gas adsorption-desorption signal can be increased. Furthermore, as heating and non-heating of the gas sensor exposed to the sample gas are alternated therebetween in the first cycle, odor molecules in the sample gas that have adhered to the gas sensor can be volatilized periodically, and the accuracy of detecting the detection-target gas can be further increased.

Furthermore, in a gas detection method according to a second aspect of the present disclosure, in the first aspect, the first cycle that is a cycle of the non-detection-target gas adsorption-desorption signal, a second cycle that is a cycle of the detection-target gas adsorption-desorption signal, and a third cycle that is the regular cycle of the reference signal may be equal to each other.

According to this aspect, a detection-target gas that occurs at a target item can be detected, for example, with the use of a single gas sensor.

Furthermore, in a gas detection method according to a third aspect of the present disclosure, in the second aspect, the gas detection method may further include (d) attenuating a direct current signal in the gas adsorption-desorption signal obtained in (b), with use of a filter having a predetermined passband.

According to this aspect, the accuracy of detecting the detection-target gas can be further increased.

Furthermore, in a gas detection method according to a fourth aspect of the present disclosure, in the first aspect, the first cycle that is a cycle of the non-detection-target gas adsorption-desorption signal, a second cycle that is a cycle of the detection-target gas adsorption-desorption signal, and a third cycle that is the regular cycle of the reference signal may be different from each other, and the first cycle may be a common multiple of the second cycle and the third cycle.

According to this aspect, a detection-target gas that occurs at a target item can be detected, for example, with the use of a plurality of gas sensors.

Furthermore, in a gas detection method according to a fifth aspect of the present disclosure, in the fourth aspect, the gas detection method may further include (d) attenuating the non-detection-target gas adsorption-desorption signal in the gas adsorption-desorption signal obtained in (b), with use of a filter having a predetermined passband.

According to this aspect, the accuracy of detecting the detection-target gas can be further increased.

Furthermore, in a gas detection method according to a sixth aspect of the present disclosure, in any one aspect of the second aspect to the fifth aspect, the gas detection method may further include (e) conveying a plurality of target items, each of which is the target item, successively to a detection region of the gas sensor in the second cycle to place each of the plurality of target items in the detection region at a timing when the gas sensor is not heated.

According to this aspect, as each of the plurality of target items is conveyed such that each of the plurality of target items is located in the detection region at a timing when the temperature of the gas sensor is relatively low (i.e., the sensitivity of the gas sensor is high), the detection-target gas that occurs at each of the plurality of target items can be detected with higher accuracy.

In a gas detection method according to a seventh aspect of the present disclosure, in the sixth aspect, the first cycle, the second cycle, and the third cycle may be determined to set a phase difference between a rise of a waveform of the detection-target gas adsorption-desorption signal and a rise of a waveform of the reference signal to 0° or 180°.

According to this aspect, the detection-target gas that flows to the gas sensor at a timing when the temperature of the gas sensor is relatively low can be detected with higher accuracy. Furthermore, in a gas detection method according to an eighth aspect of the present disclosure, in any one aspect of the first aspect to the seventh aspect, the gas detection method may further include (f) detecting the detection-target gas adsorption-desorption signal based on a multiplication result obtained by the multiplier, with use of a predetermined formula for detecting the detection-target gas adsorption-desorption signal.

According to this aspect, the use of the predetermined formula makes it possible to determine with high accuracy, for example, whether the detection-target gas is a foul-smelling gas.

A gas detection system according to a ninth aspect of the present disclosure is a gas detection system that detects a detection-target gas that occurs at a target item, and the gas detection system includes: a gas sensor that outputs a gas adsorption-desorption signal corresponding to a gas adsorption concentration; a temperature adjusting device that alternates, in a first cycle, between heating and non-heating of the gas sensor exposed to a sample gas that includes the detection-target gas and a non-detection-target gas other than the detection-target gas; an obtainer that obtains the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and a multiplier that multiplies the gas adsorption-desorption signal obtained by the obtainer and a reference signal that repeats a rise and a fall in a regular cycle.

According to this aspect, as the gas adsorption-desorption signal and the reference signal are multiplied by the multiplier, the accuracy of detecting the detection-target gas adsorption-desorption signal included in the gas adsorption-desorption signal can be increased. Furthermore, as heating and non-heating of the gas sensor exposed to the sample gas are alternated therebetween in the first cycle, odor molecules in the sample gas that have adhered to the gas sensor can be volatilized periodically, and the accuracy of detecting the detection-target gas can be further increased.

It is to be noted that general or specific aspects of the above may be implemented in the form of a system, a method, an integrated circuit, a computer program, or a computer readable recording medium, such as a CD-ROM, or may be implemented through any desired combinations of a system, a method, an integrated circuit, a computer program, and a recording medium.

Hereinafter, some embodiments will be described in specific terms with reference to the drawings.

The embodiments described below merely illustrate general or specific examples. The numerical values, the shapes, the materials, the constituent elements, the arrangement positions and the connection modes of the constituent elements, the steps, the order of the steps, and so on illustrated in the following embodiments are examples and are not intended to limit the present disclosure. Furthermore, among the constituent elements described according to the following embodiments, any constituent elements that are not cited in the independent claims expressing the broadest concepts are to construed as optional constituent elements.

### Embodiment 1

### 1-1. Overview of Gas Detection System

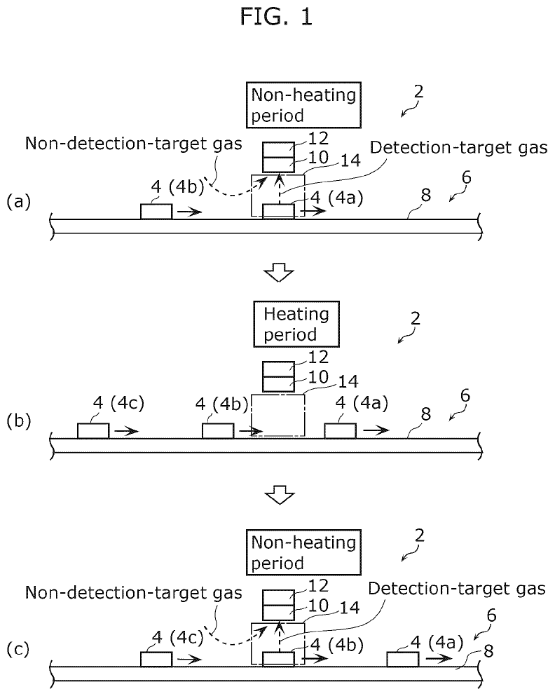

An overview of gas detection system 2 according to Embodiment 1 will be described with reference to FIG. 1. FIG. 1 is a diagram showing an overview of gas detection system 2 according to Embodiment 1.

As shown in FIG. 1, gas detection system 2 is a system for detecting a detection-target gas that occurs at target item 4 (4a, 4b, and 4c). To be more specific, target item 4 is a food item, and gas detection system 2 is a system for performing a total inspection to determine whether a foul-smelling gas (also referred to below simply as a foul odor) is occurring at target items 4 being successively conveyed in a predetermined direction (the right direction in FIG. 1) by conveyer 8 in food production line 6. In the present specification, the term “gas” means any gaseous body that contains an odor molecule.

Gas detection system 2 includes gas sensor 10 and heater 12 (one example of a temperature adjusting device). According to the present embodiment, gas detection system 2 includes only one gas sensor 10.

Gas sensor 10 is an odor sensor that outputs a gas adsorption-desorption signal corresponding to a gas adsorption concentration. Gas sensor 10 is disposed, for example, directly above conveyer 8 in food production line 6. This configuration defines detection region 14 of gas sensor 10 on conveyer 8 directly below gas sensor 10. Gas sensor 10, when exposed to a sample gas present in detection region 14, outputs a gas adsorption-desorption signal corresponding to the adsorption concentration of the sample gas.

A sample gas includes a detection-target gas and a non-detection-target gas. A detection-target gas is a gas that occurs at target item 4 conveyed to detection region 14 by conveyer 8. A non-detection-target gas refers to any gasses other than the detection-target gas (e.g., a gas in the atmosphere in food production line 6). A gas adsorption-desorption signal is a signal in which a detection-target gas adsorption-desorption signal corresponding to a detection-target gas and a non-detection-target gas adsorption-desorption signal corresponding to a non-detection-target gas are superposed on each other.

Gas sensor 10 is constituted, for example, by an electrical resistance sensor. Specifically, gas sensor 10 includes a sensing element formed by a sensitive film, and a pair of electrodes electrically connected to this sensing element. The electrical resistance value of the sensing element changes in accordance with the adsorption concentration of odor molecules in a gas that are adsorbed onto the sensing element. Gas sensor 10 outputs, via the pair of electrodes, a signal corresponding to the electrical resistance value of the sensing element in the form of a voltage signal or a current signal. Herein, gas sensor 10 is not limited to an electrical resistance sensor and may instead be constituted by any of the various sensors including, but not limited to, an electrochemical sensor, a semiconductor sensor, a field effect transistor sensor, a surface acoustic wave sensor, or a quartz crystal microbalance sensor.

Heater 12 is, for example, an electrothermal heater that produces heat through electrical resistive heating of converting supplied electric power. Heater 12 is disposed in contact with gas sensor 10 and alternates, in a first cycle (e.g., six seconds), between heating and non-heating of gas sensor 10 exposed to a sample gas. Specifically, a first cycle includes a heating period (e.g., three seconds) and a non-heating period (e.g., three seconds). In a heating period, gas sensor 10 is heated by heater 12, and thus the temperature of gas sensor 10 rises. In a non-heating period, the heating of gas sensor 10 by heater 12 is paused, and as gas sensor 10 dissipates heat, the temperature of gas sensor 10 falls. In the present specification, the term “non-heating” refers to a concept that includes not only letting gas sensor 10 dissipate heat but also actively cooling gas sensor 10.

According to the present embodiment, gas sensor 10 and heater 12 are each configured as a separate component. This, however, is not a limiting example, and gas sensor 10 and heater 12 may be configured as a single component. According to the present embodiment, heat produced by heater 12 is conducted directly to gas sensor 10, but this is not a limiting example. For example, heater 12 may heat the atmospheric gas in food production line 6, and the heat produced by heater 12 may thus be conducted indirectly to gas sensor 10 via the atmospheric gas in food production line 6. While the present embodiment uses heater 12 as a temperature adjusting device, a Peltier element may instead be used as heater 12. In this case, gas sensor 10 can be heated by the Peltier element in a heating period, and gas sensor 10 can be cooled also by the Peltier element in a non-heating period.

Due to the characteristics of gas sensor 10, odor molecules tend to adhere to gas sensor 10 more easily in a non-heating period, and thus the sensitivity of gas sensor 10 becomes higher in that period. Therefore, when target item 4 is conveyed into detection region 14 during a non-heating period, a detection-target gas that occurs at this target item 4 is detected strongly by gas sensor 10. Meanwhile, since odor molecules tend to adhere to gas sensor 10 less easily in a heating period, the sensitivity of gas sensor 10 becomes lower. Therefore, when target item 4 is conveyed into detection region 14 in a heating period, a detection-target gas that occurs at this target item 4 is detected weakly by gas sensor 10.

When gas sensor 10 is heated during a heating period, this heating allows the odor molecules that have adhered to gas sensor 10 in the non-heating period immediately preceding the heating period to volatilize and thus can clean gas sensor 10. As a result of this cleaning, more odor molecules contained in a detection-target gas can be made to adhere to gas sensor 10 in a non-heating period immediately following the heating period, and thus the accuracy of detecting the detection-target gas can be increased.

Target items 4 are conveyed by conveyer 8 to detection region 14 with a second cycle (e.g., six seconds) such that target item 4 becomes located in detection region 14 in a non-heating period (i.e., at a timing when gas sensor 10 is not heated). According to the present embodiment, the first cycle and the second cycle are equal to each other.

Now, an example in which target items 4a, 4b, and 4c are conveyed successively in this order by conveyer 8 into detection region 14 will be described.

As shown in (a) of FIG. 1, leading target item 4a is conveyed by conveyer 8 into detection region 14 in the non-heating period of the first cycle. At this point, gas sensor 10 is exposed to a sample gas (a detection-target gas and a non-detection-target gas) present in detection region 14. Through this exposure, gas detection system 2 detects a detection-target gas contained in the sample gas, that is, a detection-target gas that occurs at target item 4a located in detection region 14.

Afterward, as shown in (b) of FIG. 1, in the heating period of the first cycle, leading target item 4a is conveyed by conveyer 8 away from detection region 14, and second target item 4b is conveyed by conveyer 8 to detection region 14. Herein, the length of time from when leading target item 4a reaches detection region 14 to when second target item 4b reaches detection region 14 is equal to the second cycle.

Afterward, as shown in (c) of FIG. 1, second target item 4b is conveyed by conveyer 8 into detection region 14 in the non-heating period of the subsequent first cycle. At this point, gas sensor 10 is exposed to a sample gas present in detection region 14. Through this exposure, gas detection system 2 detects a detection-target gas contained in the sample gas, that is, a detection-target gas that occurs at target item 4b located in detection region 14.

Thereafter, in a manner similar to the one described above, gas detection system 2 detects a detection-target gas that occurs at each of target items 4a, 4b, and 4c successively conveyed by conveyer 8 into detection region 14. Through this process, if a detection-target gas that occurs at at least one of target items 4a, 4b, and 4c is determined to be a foul-smelling gas, gas detection system 2 can identify at least one of target items 4a, 4b, and 4c as a foul odor source.

### 1-2. Configuration of Gas Detection System

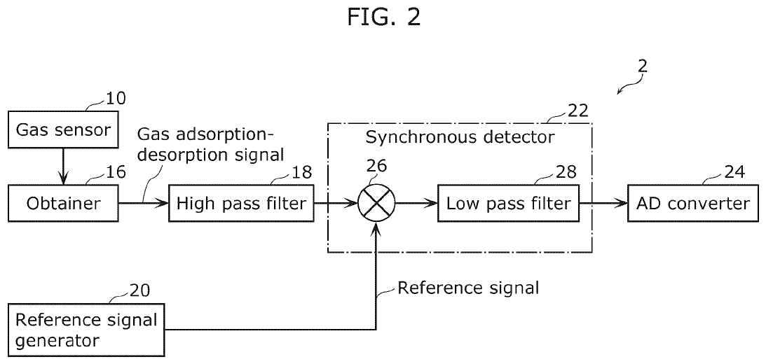

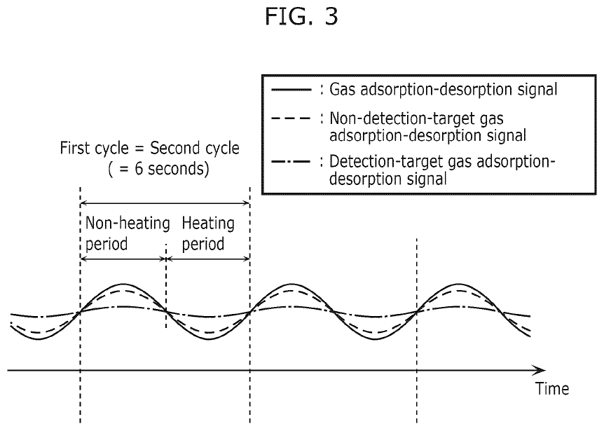

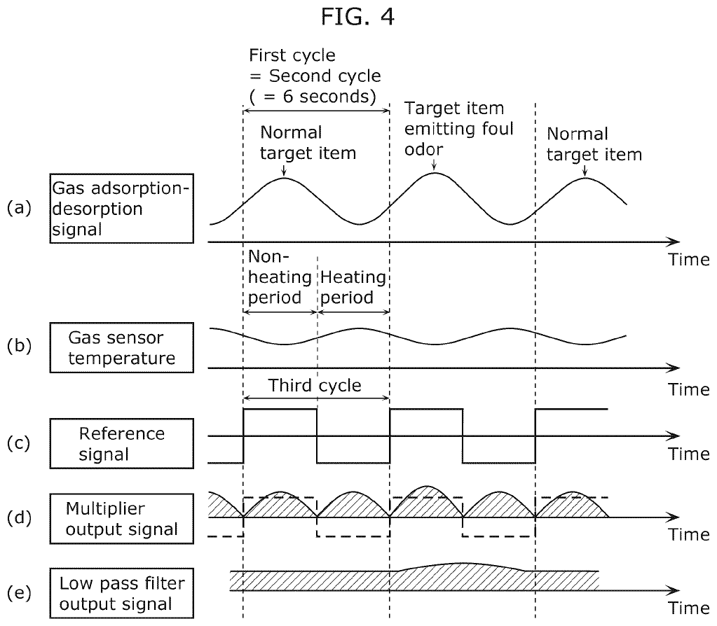

A configuration of gas detection system 2 according to Embodiment 1 will be described with reference to FIG. 2 to FIG. 4. FIG. 2 is a block diagram showing a configuration of gas detection system 2 according to Embodiment 1. FIG. 3 is a diagram showing one example of waveforms of gas adsorption-desorption signals according to Embodiment 1. FIG. 4 is a diagram showing one example of each of the waveforms of a gas adsorption-desorption signal, a gas sensor temperature, a reference signal, a multiplier output signal, and a low pass filter output signal in gas detection system 2 according to Embodiment 1. In FIG. 3 and FIG. 4, the horizontal axis represents the time, and the vertical axis represents the magnitude (the level) of each signal.

As shown in FIG. 2, gas detection system 2 includes gas sensor 10, obtainer 16, high pass filter 18, reference signal generator 20, synchronous detector 22, and AD converter 24.

Gas sensor 10 is exposed to a sample gas present in detection region 14 during the non-heating period and the heating period of the first cycle and thus outputs a gas adsorption-desorption signal corresponding to the adsorption concentration of this sample gas.

Gas sensor 10 outputs a gas adsorption-desorption signal such as the one shown in FIG. 3. As shown in FIG. 3, a gas adsorption-desorption signal (the waveform shown in the solid line) is a signal in which a detection-target gas adsorption-desorption signal (the waveform shown in the dashed-dotted line) and a non-detection-target gas adsorption-desorption signal (the waveform shown in the dashed line) are superposed on each other. The cycle of the detection-target gas adsorption-desorption signal is equal to the second cycle (e.g., six seconds), which is the cycle with which target items 4 are conveyed. Meanwhile, the cycle of the non-detection-target gas adsorption-desorption signal is equal to the first cycle (e.g., six seconds), which is the cycle with which gas sensor 10 is heated by heater 12. In other words, the cycle of the detection-target gas adsorption-desorption signal and cycle period of the non-detection-target gas adsorption-desorption signal are equal to each other. Herein, the magnitude (the amplitude) of the detection-target gas adsorption-desorption signal is smaller than the magnitude of the non-detection-target gas adsorption-desorption signal.

As shown in FIG. 3 and in (a) and (b) of FIG. 4, the magnitude of the gas adsorption-desorption signal is greater in the non-heating period than in the heating period of the first cycle. Specifically, as shown in FIG. 3, the magnitude of the detection-target gas adsorption-desorption signal is greater in the non-heating period than in the heating period of the first cycle. This is so because, as described earlier, the sensitivity of gas sensor 10 is higher in the non-heating period than in the heating period and because target item 4 is in detection region 14 and comes closest to gas sensor 10 in the non-heating period.

Furthermore, the magnitude of the non-detection-target gas adsorption-desorption signal is greater in the non-heating period than in the heating period of the first cycle. This is so because, as described earlier, the sensitivity of gas sensor 10 is higher in the non-heating period than in the heating period. Herein, as shown in (b) of FIG. 4, the temperature of gas sensor 10 held in the non-heating period is lower than the temperature of gas sensor 10 held in the heating period.

Referring back to FIG. 2, obtainer 16 obtains a gas adsorption-desorption signal output from gas sensor 10 and outputs the obtained gas adsorption-desorption signal to high pass filter 18.

High pass filter 18 is a filter having a predetermined passband. Specifically, high pass filter 18 allows a non-detection-target gas adsorption-desorption signal of the first cycle and a detection-target gas adsorption-desorption signal of the second cycle to pass therethrough and attenuates a direct current signal included in a gas adsorption-desorption signal. A gas adsorption-desorption signal that has passed through high pass filter 18 is output to synchronous detector 22. Herein, in place of high pass filter 18, a band pass filter may be used.

Reference signal generator 20 generates a reference signal and outputs the generated reference signal to synchronous detector 22.

A reference signal is a signal that repeats a rise and a fall in a regular cycle. Specifically, a reference signal is a rectangular wave with a third cycle (e.g., six seconds) such as the one shown in (c) of FIG. 4. According to the present embodiment, the first cycle, the second cycle, and the third cycle are equal to each other. A reference signal is at high level during a non-heating period and is at low level during a heating period. Herein, the phase difference between the rise of the waveform of the detection-target gas adsorption-desorption signal (i.e., the waveform of the gas adsorption-desorption signal) and the rise of the waveform of the reference signal is set to 0°, and the first cycle, the second cycle, and the third cycle are all set to the same cycle (e.g., six seconds).

According to the present embodiment, the phase difference between the rise of the waveform of the detection-target gas adsorption-desorption signal and the rise of the waveform of the reference signal is set to 0°. This, however, is not a limiting example, and this phase difference may be set to 180°. In this case, the reference signal is at low level during a non-heating period and is at high level during a heating period. Furthermore, according to the present embodiment, the reference signal is a rectangular wave. This, however, is not a limiting example, and the reference signal may be, for example, a sinusoidal wave.

Synchronous detector 22 is a so-called lock-in amplifier and includes multiplier 26 and low pass filter 28.

Multiplier 26 is constituted, for example, by a field programmable gate array (FPGA) and multiplies a gas adsorption-desorption signal from high pass filter 18 and a reference signal from reference signal generator 20. Through this multiplication, as shown in (d) of FIG. 4, a modulated signal (also referred to below as a multiplier output signal) is obtained in which, of the gas adsorption-desorption signal from high pass filter 18, only the frequency component at the same frequency as the reference signal is converted to a direct current signal and the remaining frequency components are converted to an alternating current signal. Multiplier 26 outputs the multiplier output signal obtained as a result of the multiplication to low pass filter 28.

Low pass filter 28 is a filter having a predetermined passband. Specifically, low pass filter 28, of the multiplier output signal from multiplier 26, allows only the direct current signal to pass therethrough and attenuates the alternating current signal. With this configuration, as shown in (e) of FIG. 4, a signal in which, of the gas adsorption-desorption signal, only the frequency component at the same frequency as the reference signal is extracted (also referred to below as a low pass filter output signal) is output from low pass filter 28. Low pass filter 28 outputs the low pass filter output signal to AD converter 24.

AD converter 24 performs analog-to-digital (AD) conversion on the low pass filter output signal from low pass filter 28 to convert the low pass filter output signal from an analog signal to a digital signal. Through this operation, with the use of a predetermined formula for detecting a detection-target gas adsorption-desorption signal, a detection-target gas adsorption-desorption signal can be detected based on the low pass filter output signal converted to a digital signal (i.e., based on the multiplication result of multiplier 26). Herein, a predetermined formula means a computation model that is based on an algorithm using machine learning.

For example, as shown in (a) of FIG. 4, the magnitude of the gas adsorption-desorption signal detected when target item 4 emitting a foul-smelling gas is located in detection region 14 is greater than the magnitude of the gas adsorption-desorption signal detected when normal target item 4 emitting no foul-smelling gas is located in detection region 14. In accordance with the above, as shown in (e) of FIG. 4, the magnitude of the low pass filter output signal detected when target item 4 emitting a foul-smelling gas is located in detection region 14 is greater than the magnitude of the low pass filter output signal detected when normal target item 4 is located in detection region 14. Monitoring such a change in the magnitude of the low pass filter output signal makes it possible to determine whether a foul-smelling gas is being emitted from any of a plurality of target items 4 successively conveyed by conveyer 8 in food production line 6 (see FIG. 1).

### 1-3. Operation of Gas Detection System

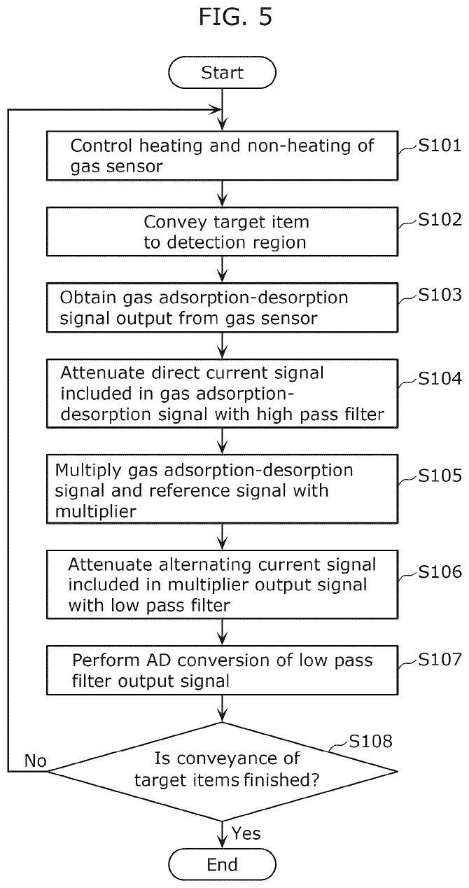

An operation of gas detection system 2 according to Embodiment 1 will be described with reference to FIG. 5. FIG. 5 is a flowchart showing a flow of an operation of gas detection system 2 according to Embodiment 1.

As shown in FIG. 5, heater 12 controls heating and non-heating of gas sensor 10 (S101). Specifically, heater 12 alternates, in a first cycle, between heating and non-heating of gas sensor 10 exposed to a sample gas.

Target item 4 is conveyed to detection region 14 by conveyer 8 in food production line 6 (S102). Thus, gas sensor 10, when exposed to the sample gas present in detection region 14, outputs a gas adsorption-desorption signal corresponding to the adsorption concentration of the sample gas.

Obtainer 16 obtains the gas adsorption-desorption signal output from gas sensor 10 (S103) and outputs the obtained gas adsorption-desorption signal to high pass filter 18.

High pass filter 18 allows a non-detection-target gas adsorption-desorption signal and a detection-target gas adsorption-desorption signal included in the gas adsorption-desorption signal to pass therethrough and attenuates a direct current signal included in the gas adsorption-desorption signal (S104). The gas adsorption-desorption signal that has passed through high pass filter 18 is output to multiplier 26 of synchronous detector 22.

Multiplier 26 multiplies the gas adsorption-desorption signal from high pass filter 18 and a reference signal from reference signal generator 20 (S105). Multiplier 26 outputs a multiplier output signal obtained as a result of the multiplication to low pass filter 28.

Low pass filter 28, of the multiplier output signal from multiplier 26, allows only the direct current signal to pass therethrough and attenuates the alternating current signal (S106). Low pass filter 28 outputs the low pass filter output signal to AD converter 24.

AD converter 24 performs AD conversion on the low pass filter output signal from low pass filter 28 to convert the low pass filter output signal from an analog signal to a digital signal (S107).

If the conveyance of target items 4 is to be continued (NO at S108), the process returns to step S101 described above. Meanwhile, if the conveyance of target items 4 is to be ended (YES at S108), the operation in the flowchart shown in FIG. 5 is terminated.

### 1-4. Advantageous Effects

As described above, according to the present embodiment, as a gas adsorption-desorption signal and a reference signal are multiplied by multiplier 26 of synchronous detector 22, signal components derived from the odors (e.g., constantly present odors in the atmosphere in food production line 6, odors of people approaching gas sensor 10, etc.) that are not synchronous with the conveyance cycle of target items 4 (the second cycle) can be reduced.

Furthermore, influence of white noise, 1/f noise, noise from the commercial power source (50 to 60 Hz), or any other electro-magnetic compatibility (EMC) noise can be greatly reduced. As a result, the present embodiment makes it possible to determine with high accuracy whether a foul-smelling gas is occurring at object target item 4.

### Embodiment 2

### 2-1. Overview of Gas Detection System

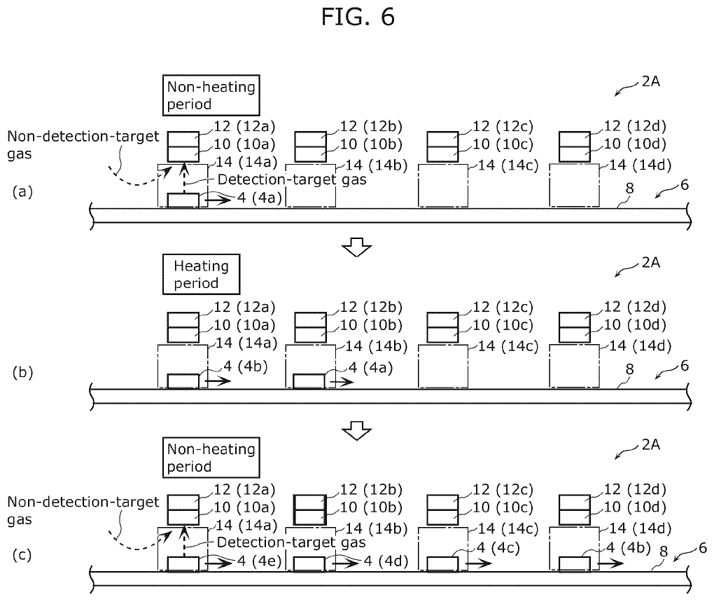

An overview of gas detection system 2A according to Embodiment 2 will be described with reference to FIG. 6. FIG. 6 is a diagram showing an overview of gas detection system 2A according to Embodiment 2. In the description of the present embodiment, constituent elements identical to those in Embodiment 1 described above will be given identical reference characters, and description thereof will be omitted.

As shown in FIG. 6, gas detection system 2A according to Embodiment 2 includes four gas sensors 10 (10a, 10b, 10c, and 10d) and four heaters 12 (12a, 12b, 12c, and 12d).

Four gas sensors 10 (10a to 10d) are disposed in this order with a space therebetween along the conveyance path of conveyer 8. Herein, gas sensor 10a is disposed at the furthest upstream in the conveyance path of conveyer 8, and gas sensor 10d is disposed at the furthest downstream in the conveyance path of conveyer 8. On conveyer 8, four detection regions 14 (14a, 14b, 14c, and 14d) are defined directly below four respective gas sensors 10 (10a to 10d). Four heaters 12 (12a to 12d) are disposed in contact with four respective gas sensors 10 (10a to 10d), and four heaters 12 (12a to 12d) each alternate, in a first cycle (e.g., six seconds), between heating and non-heating of the corresponding one of gas sensors 10 (10a to 10d) exposed to a sample gas.

Herein, the heating periods in which four respective gas sensors 10 (10a to 10d) are heated by four respective heaters 12 (12a to 12d ) are temporally off from each other by a predetermined time. In other words, the timing at which the heating period in which gas sensor 10a is heated by heater 12a starts precedes, by the predetermined time, the timing at which the heating period in which gas sensor 10b is heated by heater 12b starts. Furthermore, the timing at which the heating period in which gas sensor 10b is heated by heater 12b starts precedes, by the predetermined time, the timing at which the heating period in which gas sensor 10c is heated by heater 12c starts. Additionally, the timing at which the heating period in which gas sensor 10c is heated by heater 12c starts precedes, by the predetermined time, the timing at which the heating period in which gas sensor 10d is heated by heater 12d starts.

Target items 4 (4a, 4b, 4c, 4d, and 4e) are each conveyed by conveyer 8 into any of four detection regions 14a to 14d with a second cycle (e.g., 1.5 seconds) such that target items 4 (4a, 4b, 4c, 4d, and 4e) are each located in any of four detection regions 14a to 14d in a non-heating period. According to the present embodiment, the second cycle is shorter than the first cycle.

The following description focuses on the workings of gas sensor 10a alone observed while target items 4a, 4b, 4c, 4d, and 4e are conveyed successively in this order by conveyer 8 into detection region 14a.

As shown in (a) of FIG. 6, leading target item 4a is conveyed by conveyer 8 into detection region 14a in the non-heating period of the first cycle, which is the period with which gas sensor 10a is heated.

At this point, gas sensor 10a becomes exposed to a sample gas (a detection-target gas and a non-detection-target gas) present in detection region 14a. Through this exposure, gas detection system 2A detects the detection-target gas contained in the sample gas, that is, the detection-target gas that has occurred at target item 4a located in detection region 14a.

Afterward, as shown in (b) of FIG. 6, in the heating period of the first cycle, which is the period with which gas sensor 10a is heated, leading target item 4a is conveyed by conveyer 8 away from detection region 14a, and second target item 4b is conveyed by conveyer 8 to detection region 14a. Herein, the length of time from when leading target item 4a reaches detection region 14a to when second target item 4b reaches detection region 14a is equal to the second cycle.

Afterward, as shown in (c) of FIG. 6, fifth target item 4e is conveyed by conveyer 8 into detection region 14a in the non-heating period of the next first cycle, which is the period with which gas sensor 10a is heated. At this point, gas sensor 10a becomes exposed to the sample gas present in detection region 14a. Through this exposure, gas detection system 2A detects the detection-target gas contained in the sample gas, that is, the detection-target gas that has occurred at target item 4e located in detection region 14a.

The description above has focused on the workings of gas sensor 10a alone, but gas sensors 10b to 10d also work in a similar manner to gas sensor 10a described above. In other words, the detection-target gas that has occurred at each of target items 4b, 4c, and 4d, other than target items 4a and 4e, in (a) to (c) of FIG. 6 becomes the target of detection of any one of gas sensors 10b to 10d when target items 4b, 4c, and 4d are conveyed into any of detection regions 14b to 14d.

### 2-2. Configuration of Gas Detection System

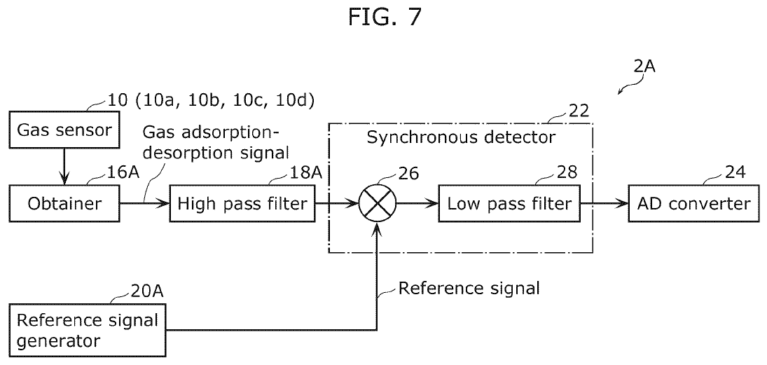

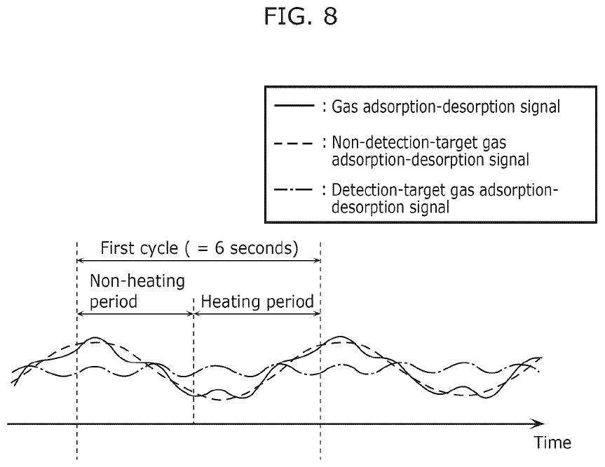

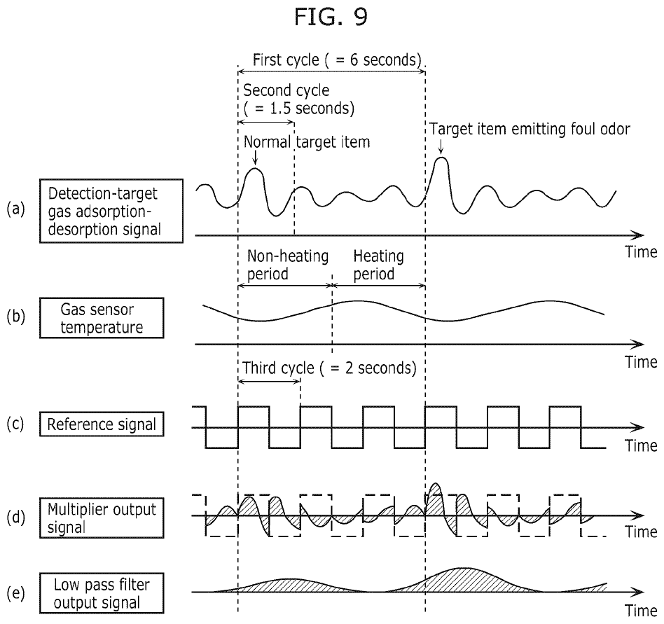

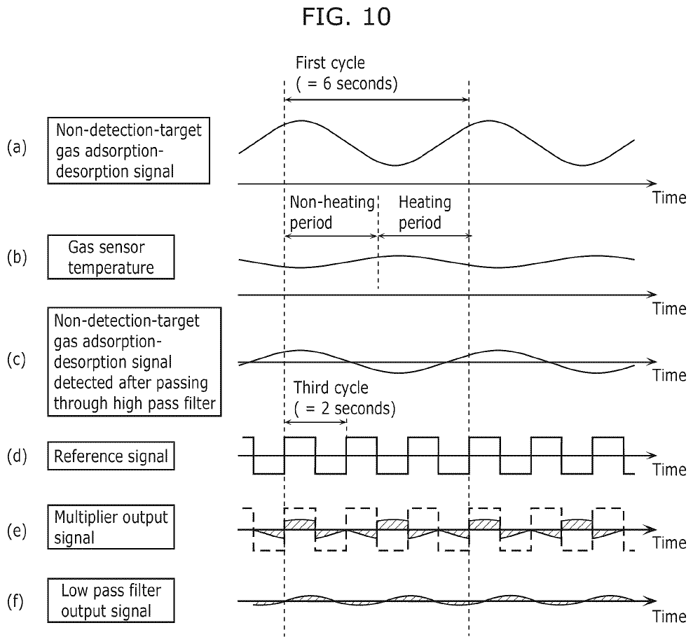

A configuration of gas detection system 2A according to Embodiment 2 will be described with reference to FIG. 7 to FIG. 10. FIG. 7 is a block diagram showing a configuration of gas detection system 2A according to Embodiment 2. FIG. 8 is a diagram showing one example of waveforms of gas adsorption-desorption signals according to Embodiment 2. FIG. 9 is a diagram showing one example of each of the waveforms of a detection-target gas adsorption-desorption signal, a gas sensor temperature, a reference signal, a multiplier output signal, and a low pass filter output signal in gas detection system 2A according to Embodiment 2. FIG. 10 is a diagram showing one example of each of the waveforms of a non-detection-target gas adsorption-desorption signal, a gas sensor temperature, a non-detection-target gas adsorption-desorption signal observed after the signal passes through a high pass filter, a reference signal, a multiplier output signal, and a low pass filter output signal in gas detection system 2A according to Embodiment 2.

As shown in FIG. 7, gas detection system 2A includes gas sensors 10 (10a to 10d), obtainer 16A, high pass filter 18A, reference signal generator 20A, synchronous detector 22, and AD converter 24.

Gas sensors 10 (10a to 10d) each output a gas adsorption-desorption signal such as the one shown in FIG. 8. As shown in FIG. 8, a gas adsorption-desorption signal (the waveform shown in the solid line) is a signal in which a detection-target gas adsorption-desorption signal (the waveform shown in the dashed-dotted line) and a non-detection-target gas adsorption-desorption signal (the waveform shown in the dashed line) are superposed on each other. The period of the detection-target gas adsorption-desorption signal is equal to the second cycle (e.g., 1.5 seconds), which is the period with which target items 4 are conveyed. Meanwhile, the period of the non-detection-target gas adsorption-desorption signal is equal to the first cycle (e.g., six seconds), which is the period with which gas sensors 10 (10a to 10d) are heated by heaters 12 (12a to 12d). In other words, the period of the detection-target gas adsorption-desorption signal is shorter than the period of the non-detection-target gas adsorption-desorption signal. Herein, a reason why the first cycle is set longer than the second cycle is that it may be difficult to set the period with which gas sensors 10 (10a to 10d) are heated as short as the period with which target items 4 are conveyed.

In the interest of making the description easier to understand, FIG. 9 shows only each of the waveforms pertaining to the detection-target gas adsorption-desorption signal of the gas adsorption-desorption signal, and FIG. 10 shows only each of the waveforms pertaining to the non-detection-target gas adsorption-desorption signal of the gas adsorption-desorption signal. Furthermore, FIG. 9 and FIG. 10 show each of the waveforms pertaining to the gas adsorption-desorption signal output from gas sensor 10a.

As shown in (a) and (b) of FIG. 9, the magnitude of the detection-target gas adsorption-desorption signal is greater in the non-heating period than in the heating period of the first cycle. Furthermore, as shown in (a) of FIG. 9, the magnitude of the detection-target gas adsorption-desorption signal becomes even greater during the period from the beginning of the first cycle until the time corresponding to the second cycle passes. This is so because, during this period, the sensitivity of gas sensor 10a becomes higher as the temperature of gas sensor 10a is lower in the non-heating period and because target item 4 comes closest to gas sensor 10a. Furthermore, as shown in (a) and (b) of FIG. 10, the magnitude of the non-detection-target gas adsorption-desorption signal is greater in the non-heating period than in the heating period of the first cycle.

Obtainer 16A obtains a gas adsorption-desorption signal output from each of gas sensors 10 (10a to 10d) and outputs the obtained gas adsorption-desorption signals to high pass filter 18A. Thereafter, the multiplication process by synchronous detector 22 and other processes are executed on each of the gas adsorption-desorption signals output from gas sensors 10 (10a to 10d).

High pass filter 18 allows a detection-target gas adsorption-desorption signal of the second cycle to pass therethrough, attenuates a non-detection-target gas adsorption-desorption signal of the first cycle, and attenuates a direct current signal included in a gas adsorption-desorption signal. With this configuration, the magnitude of the non-detection-target gas adsorption-desorption signal is attenuated, as shown in (a) and (c) of FIG. 10. Herein, high pass filter 18A may be constituted, for example, by a digital filter that obtains the derivative value with an FPGA.

Reference signal generator 20A generates a reference signal and outputs the generated reference signal to synchronous detector 22. A reference signal is a rectangular wave with a third cycle (e.g., two seconds) such as the one shown in (c) of FIG. 9 or (d) of FIG. 10. According to the present embodiment, the first cycle, the second cycle, and the third cycle are different from each other, and the first cycle is a common multiple of the second cycle and the third cycle. Herein, the phase difference between the rise of the waveform of the detection-target gas adsorption-desorption signal (i.e., the waveform of the gas adsorption-desorption signal) and the rise of the waveform of the reference signal is set to 0°, and the first cycle, the second cycle, and the third cycle are all set to different periods. According to the present embodiment, the phase difference between the rise of the waveform of the detection-target gas adsorption-desorption signal and the rise of the waveform of the reference signal is set to 0°. This, however, is not a limiting example, and this phase difference may be set to 180°.

Multiplier 26 of synchronous detector 22 multiplies a gas adsorption-desorption signal from high pass filter 18A and a reference signal from reference signal generator 20A. Through this multiplication, as shown in (d) of FIG. 9, a modulated signal is obtained in which, of the detection-target gas adsorption-desorption signal included in the gas adsorption-desorption signal from high pass filter 18A, only the frequency component at the same frequency as the reference signal is converted to a direct current signal and the remaining frequency components are converted to an alternating current signal. Furthermore, as shown in (e) of FIG. 10, a modulated signal is obtained in which, of the non-detection-target gas adsorption-desorption signal included in the gas adsorption-desorption signal from high pass filter 18A, only the frequency component at the same frequency as the reference signal is converted to a direct current signal and the remaining frequency components are converted to an alternating current signal. Multiplier 26 outputs the multiplier output signal obtained as a result of the multiplication to low pass filter 28. Herein, the multiplier output signal is a signal in which the multiplier output signal shown in (d) of FIG. 9 and the multiplier output signal shown in (e) of FIG. 10 are added together.

Low pass filter 28, of the multiplier output signal from multiplier 26 that is shown in (d) of FIG. 9, allows only the direct current signal to pass therethrough and attenuates the alternating current signal. Furthermore, low pass filter 28, of the multiplier output signal from multiplier 26 that is shown in (e) of FIG. 10, allows only the direct current signal to pass therethrough and attenuates the alternating current signal. Through this operation, low pass filter 28 outputs a low pass filter output signal to AD converter 24. Herein, the low pass filter output signal is a signal in which the low pass filter output signal shown in (e) of FIG. 9 and the low pass filter output signal shown in (f) of FIG. 10 are added together.

### 2-3. Operation of Gas Detection System

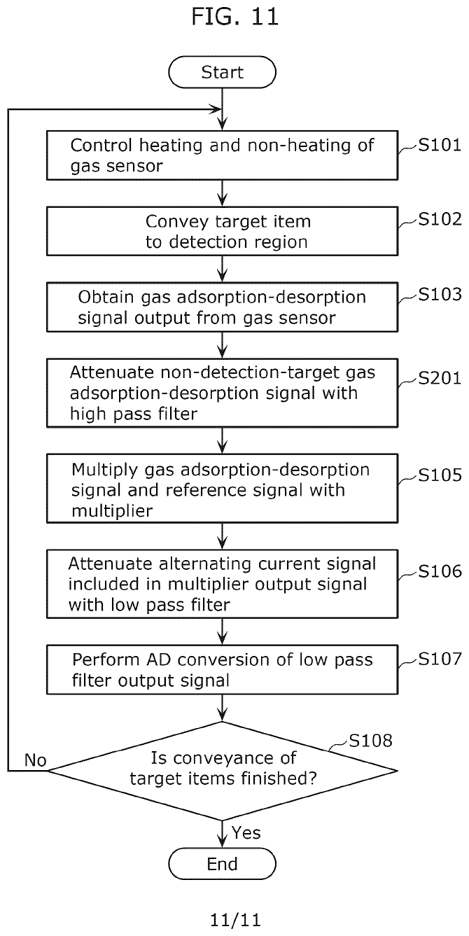

An operation of gas detection system 2A according to Embodiment 2 will be described with reference to FIG. 11. FIG. 11 is a flowchart showing a flow of an operation of gas detection system 2A according to Embodiment 2. In the flowchart shown in FIG. 11, processes identical to those in FIG. 5 according to Embodiment 1 described above are given identical step numbers, and description thereof will be omitted.

As shown in FIG. 11, steps S101 to S103 are executed in manners similar to those according to Embodiment 1 described above. After step S103, high pass filter 18A allows the detection-target gas adsorption-desorption signal included in the gas adsorption-desorption signal to pass therethrough and attenuates the non-detection-target gas adsorption-desorption signal included in the gas adsorption-desorption signal (S201). Thereafter, steps S105 to S108 are executed in manners similar to those according to Embodiment 1 described above.

### 2-4. Advantageous Effects

As described above, according to the present embodiment, a gas adsorption-desorption signal and a reference signal are multiplied by multiplier 26 of synchronous detector 22. Through this multiplication, signal components derived from the odors (e.g., constantly present odors in the atmosphere in food production line 6, odors of people approaching gas sensor 10, etc.) that are not synchronous with the conveyance period of target items 4 (the second cycle) can be reduced. Furthermore, influence of white noise, 1/f noise, noise from the commercial power source (50 to 60 Hz), or any other EMC noise can be greatly reduced. Moreover, signal components derived from odors of other target items 4 (e.g., target item 4b) conveyed before or after object target item 4 (e.g., target item 4a) can be reduced. As a result, the present embodiment makes it possible to determine with high accuracy whether a foul-smelling gas is occurring at object target item 4.

### Variations and Others

Thus far, gas detection systems according to one or more aspects have been described based on the foregoing embodiments, but these embodiments do not limit the present disclosure. Unless departing from the spirit of the present disclosure, an embodiment obtained by making various modifications that a person skilled in the art can conceive of to any of the foregoing embodiments or an embodiment constructed by combining constituent elements in different embodiments may also be encompassed by the scope of the one or more aspects.

The number of gas sensors 10 is one or four according to the foregoing embodiments, but this number is not limited thereto, and the number of gas sensors 10 may be set as desired, such as to two, three, or five or more.

In the foregoing embodiments, the constituent elements may each be implemented by dedicated hardware or may each be implemented through execution of a software program suitable for a corresponding constituent element. Each of the constituent elements may be implemented as a program executing unit, such as a CPU or a processor, reads out a software program recorded in a recording medium, such as a hard disk or a semiconductor memory, and executes the software program.

Part or the whole of the functions of the gas detection systems according to the foregoing embodiments may be implemented as a processor, such as a CPU, executes a program.

Part or the whole of the constituent elements constituting each of the devices described above may be implemented by an IC card that can be attached to or detached from the device or by a stand-alone module. Such an IC card or a module is a computer system constituted by a microprocessor, a ROM, a RAM, and so on. The IC card or the module may include an ultra-multifunctional LSI circuit. The IC card or the module implements its functions as the microprocessor operates in accordance with a computer program. The IC card or the module may be tamper-resistant.

The present disclosure may be implemented in the form of any of the methods described above. Furthermore, the present disclosure may be implemented in the form of a computer program that implements any of these methods by a computer or in the form of digital signals composed of such a computer program. The present disclosure may also be implemented in the form of a computer readable non-transitory recording medium having the aforementioned computer program or digital signals recorded thereon, and examples of such a computer readable non-transitory recording medium include a flexible disk, a hard disk, a CD-ROM, an MO, a DVD, a DVD-ROM, a DVD-RAM, a Blue-ray (registered trademark) disc (BD), and a semiconductor memory. Moreover, the present disclosure may be implemented in the form of the digital signals recorded on any of the aforementioned recording media. The present disclosure may also be implemented as the aforementioned computer program or digital signals are transmitted via an electric communication circuit, a wireless or wired communication circuit, a network represented by the internet, data broadcast, or the like. The present disclosure may also be implemented in the form of a computer system provided with a microprocessor and a memory. The memory may store the aforementioned computer program, and the microprocessor may operate in accordance with that computer program. The present disclosure may also be implemented as the aforementioned program or digital signals recorded on any of the aforementioned recording media are transported, or as the aforementioned program or digital signals are transported via any of the aforementioned networks or the like, and as the program or the digital signals are executed by a separate, independent computer system.

### INDUSTRIAL APPLICABILITY

The gas detection method according to the present disclosure is useful in, for example, a system for inspecting the presence of a foul odor in a food item, for example, in a food production line.

### REFERENCE SIGNS LIST

2, 2A gas detection system
4, 4a, 4b, 4c, 4d, 4e target item
6 food production line
8 conveyer
10, 10a, 10b, 10c, 10d gas sensor
12, 12a, 12b, 12c, 12d heater
14, 14a, 14b, 14c, 14d detection region
16, 16A obtainer
18, 18A high pass filter
20, 20A reference signal generator
22 synchronous detector
24 AD converter
26 multiplier
28 low pass filter

1. A gas detection method of detecting a detection-target gas that occurs at a target item, with use of a gas sensor that outputs a gas adsorption-desorption signal corresponding to a gas adsorption concentration, the gas detection method comprising:
(a) alternating, in a first cycle, heating and non-heating of the gas sensor exposed to a sample gas that includes the detection-target gas and a non-detection-target gas other than the detection-target gas;
(b) obtaining the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and
(c) multiplying, with use of a multiplier, the gas adsorption-desorption signal obtained in (b) and a reference signal that repeats a rise and a fall in a regular cycle.

2. The gas detection method according to claim 1, wherein
the first cycle that is a cycle of the non-detection-target gas adsorption-desorption signal, a second cycle that is a cycle of the detection-target gas adsorption-desorption signal, and a third cycle that is the regular cycle of the reference signal are equal to each other.

3. The gas detection method according to claim 2, further comprising:
(d) attenuating a direct current signal in the gas adsorption-desorption signal obtained in (b), with use of a filter having a predetermined passband.

4. The gas detection method according to claim 1, wherein
the first cycle that is a cycle of the non-detection-target gas adsorption-desorption signal, a second cycle that is a cycle of the detection-target gas adsorption-desorption signal, and a third cycle that is the regular cycle of the reference signal are different from each other, and
the first cycle is a common multiple of the second cycle and the third cycle.

5. The gas detection method according to claim 4, further comprising:
(d) attenuating the non-detection-target gas adsorption-desorption signal in the gas adsorption-desorption signal obtained in (b), with use of a filter having a predetermined passband.

6. The gas detection method according to claim 2, further comprising:
(e) conveying a plurality of target items, each of which is the target item, successively to a detection region of the gas sensor in the second cycle to place each of the plurality of target items in the detection region at a timing when the gas sensor is not heated.

7. The gas detection method according to claim 6, wherein
the first cycle, the second cycle, and the third cycle are determined to set a phase difference between a rise of a waveform of the detection-target gas adsorption-desorption signal and a rise of a waveform of the reference signal to 0° or 180°.

8. The gas detection method according to claim 1, further comprising:
(f) detecting the detection-target gas adsorption-desorption signal based on a multiplication result obtained by the multiplier, with use of a predetermined formula for detecting the detection-target gas adsorption-desorption signal.

9. A gas detection system that detects a detection-target gas that occurs at a target item, the gas detection system comprising:
a gas sensor that outputs a gas adsorption-desorption signal corresponding to a gas adsorption concentration;
a temperature adjusting device that alternates, in a first cycle, between heating and non-heating of the gas sensor exposed to a sample gas that includes the detection-target gas and a non-detection-target gas other than the detection-target gas;
an obtainer that obtains the gas adsorption-desorption signal output from the gas sensor, the gas adsorption-desorption signal being a signal in which a detection-target gas adsorption-desorption signal corresponding to the detection-target gas and a non-detection-target gas adsorption-desorption signal corresponding to the non-detection-target gas are superposed on each other; and
a multiplier that multiplies the gas adsorption-desorption signal obtained by the obtainer and a reference signal that repeats a rise and a fall in a regular cycle.
